(19) United States
(12) Patent Application Publication
(10) Pub. No.: US 2026/0210845 A1
(43) Pub. Date: Jul. 23, 2026

(54) DEVICE AND METHOD FOR HARMFUL GAS DETECTION IN TUNNEL ADVANCE DRILLING

(71) Applicant: CHINA MCC5 GROUP CORP.LTD, Sichuan (CN)

(72) Inventors: Yansong Li, Sichuan (CN); Li Luo, Sichuan (CN); Xianzhi Wang, Sichuan (CN); Weihua Liu, Sichuan (CN); Yougui Li, Sichuan (CN); Junjie Yang, Sichuan (CN)

(73) Assignee: CHINA MCC5 GROUP CORP.LTD, Sichuan (CN)

(21) Appl. No.: 19/255,958

(22) Filed: Jun. 30, 2025

(30) Foreign Application Priority Data
Jan. 22, 2025 (CN) 202510100207.4

(51) Int. Cl. G01N 21/31; E21F 17/00; G01C 3/02; G01N 33/00

(52) U.S. Cl. CPC G01N 21/31; E21F 17/00; G01C 3/02; G01N 33/0027

(57) ABSTRACT

The present application discloses a device and method for harmful gas detection in tunnel advance drilling, which relates to the technical field of tunnel construction. The device for harmful gas detection in tunnel advance drilling comprises a casing pipe, and the casing pipe is provided with a laser monitoring module, used for emitting laser axially in the casing pipe, ranging, and monitoring harmful gas concentration; the casing pipe is provided with an occlusion module for periodically blocking laser emitted by the laser monitoring module; and the casing pipe also includes a gas flow rate monitoring module for monitoring the flow rate of harmful gas within the casing pipe. The present application enables accurate detection of harmful gas concentration at the bottom of advance drill hole, providing reliable data for risk assessment of harmful gas in the tunnel.

### CROSS-REFERENCE TO RELATED APPLICATION

This application claims the priority benefit of China application serial no. 202510100207.4, filed on Jan. 22, 2025. The entirety of the above-mentioned patent application is hereby incorporated by reference herein and made a part of this specification.

### BACKGROUND

### Technical Field

The present application relates to the technical field of tunnel construction, in particular to a device and method for harmful gas detection in tunnel advance drilling.

During tunnel construction, harmful gases often escape or gush from the tunnel face or unsupported tunnel walls. When harmful gas concentration reaches certain levels, it may cause disasters threatening safety of operators, delay in construction, or even major construction accident.

To mitigate the risk of harmful gases in tunnel construction, advance geological forecast method is typically employed before formal excavation of the tunnel face, to determine the hazards of harmful gases in the tunnel.

The existing methods for advanced detection of harmful gas in tunnel mainly include two methods: The first method involves installing sensors at drill hole opening to measure harmful gas concentration; however, it cannot accurately detect harmful gas concentration in the hole. The second method involves sealing drill holes for 24 hours after completion and then measuring the harmful gas concentration in the hole; yet this method fails to detect the actual harmful gas concentration during excavation while causing low construction efficiency and hindering normal tunneling progress.

### SUMMARY

The objective of the present application is to provide a device and method for harmful gas detection in tunnel advance drilling that solves the problem of inaccurate gas concentration measurement in advance drilling hole.

The technical solutions adopted by the present application to solve its technical problem are as follows:

In the first aspect, the invention provides a device for harmful gas detection in tunnel advance drilling, which comprises a casing pipe, and the casing pipe is provided with a laser monitoring module, which is used for emitting laser axially in the casing pipe, ranging and monitoring harmful gas concentration; the casing pipe is provided with an occlusion module for periodically blocking laser emitted by the laser monitoring module; and the casing pipe is provided with a gas flow rate monitoring module for monitoring the velocity of harmful gas in the casing pipe.

Furthermore, the laser monitoring module includes a laser gas sensor, a laser ranging sensor and a reflecting prism mounted outside the casing pipe; the casing pipe wall features a laser hole, which reflects laser beams emitted by the laser gas sensor and the laser ranging sensor through the laser hole for axial propagation inside and along the casing pipe.

Furthermore, the laser monitoring module incorporates a monitoring box connected to the casing pipe, housing the laser gas sensor, laser ranging sensor, and reflecting prism.

Furthermore, the occlusion module comprises a driver outside the casing pipe and an occluder connected with the casing pipe, and an avoidance hole for dodging the occluder on the wall of the casing pipe; and the driver is used for driving movement of the occluder and periodically occlude laser in the casing pipe.

Furthermore, the gas flow rate monitoring module consists of a flow rate monitoring box outside the casing pipe and a gas flow rate sensor in the velocity monitoring box, with the velocity monitoring box connected to the cavity of the casing pipe.

Furthermore, a control module is included, which is connected to the laser monitoring module, the occlusion module, and the gas flow rate monitoring module.

Furthermore, the casing pipe is put on a drill rod, and moves axially along the drill rod.

Furthermore, a water tank is arranged below the casing pipe interconnected with its cavity; the bottom of the cavity of the water tank is arranged with a water barrier extending upward, forming an outlet chamber and a sampling chamber on both sides of the water barrier. The lower end of the water tank is provided with an outlet pipe connected to the water outlet chamber, and a sampling pipe connected to the sampling chamber.

Furthermore, the water tank includes a horizontal filter above the water barrier in the water tank, with a slag discharge door above the water tank for opening/closing the cavity above the filter.

In the second aspect, the invention provides a device for harmful gas detection in tunnel advance drilling, using the harmful gas detection device for advance drilling in tunnel provided in the First Aspect, which comprises:
S1: Connecting one end of the casing pipe to the advance drilling hole opening;
S2: The laser monitoring module emits laser beam to the bottom of the advance drilling hole, and obtains the harmful gas concentration cl from the laser monitoring module to the bottom of the advance drilling hole.
S3: After a time interval tl, the occlusion module blocks laser beam inside the casing pipe, and the laser monitoring module emits laser beam to the occlusion module and obtains the harmful gas concentration c2 and the distance d2 from the laser monitoring module to the occlusion module. The gas flow rate monitoring module monitors the flow rate v of harmful gas inside the casing pipe.
S4: After a time interval t2, the occlusion module releases laser beam inside the casing pipe, the laser monitoring module emits laser beam to the bottom of the advance drilling hole and obtains the harmful gas concentration c3 from the laser monitoring module to the bottom of the advance drilling hole.
S5: The harmful gas concentration at the bottom of the advance drilling hole is calculated based on the data obtained in Steps S2, S3 and S4.

The beneficial effects of the present application are as follows:

The device and method for harmful gas detection in tunnel advance drilling provided by the embodiments of the present application enable real-time and accurate detection of harmful gas concentration at the bottom of advance drilling hole during drilling operations, to improve construction efficiency and provide precise data for hazard assessment of harmful gases in tunnel.

### BRIEF DESCRIPTION OF THE DRAWINGS

To better illustrate the technical solutions in the embodiments of the present application, drawings used in the embodiments are briefly described below. It should be understood that the drawings described below only show some embodiments of the present application, and shall not be construed as restrictions to the scope. Those of ordinary skill in the art may also obtain other drawings on the basis of those drawings without creative work.

FIG. 1 shows a perspective view of the device for harmful gas detection in tunnel advance drilling according to the present embodiment;

FIG. 2 shows a sectional view of the device for harmful gas detection in tunnel advance drilling according to the present embodiment;

FIG. 3 shows a partial enlarged view of the device for harmful gas detection in tunnel advance drilling according to the present embodiment;

FIG. 4 shows a state diagram of drilling with the device for harmful gas detection in tunnel advance drilling according to the present embodiment.

### DESCRIPTION OF THE EMBODIMENTS

The technical solutions in the embodiments of the present application will be clearly and completely described below in conjunction with the drawings in the embodiments of the present application. Apparently, the embodiments described are only part of the embodiments, not all of the embodiments of the present application. Based on the embodiments herein, all other embodiments obtained by those of ordinary skill in the art without any creative work also fall into the protection scope of the application. The embodiments in the present application and the features in the embodiments may be combined with each other without conflict.

For description of the present application, it should be noted that the direction or position relations indicated by the terms “above”, “under”, “left”, “right”, “front”, “rear”, “inside”, “outside”, etc. are based on the orientation or position relations shown in the drawings, only to facilitate description of the present invention and simplified description, but not to indicate or imply that the indicated device or element must have a specific orientation and be constructed and operated in a specific orientation. Therefore, they shall not be construed as a limitation hereto. Unless otherwise specified, the above directional descriptions may be flexibly set during practical application as long as the relative positional relationships shown in the drawings are maintained.

For description of the present application, it should be noted that unless otherwise explicitly specified and defined, the terms “arrange,” “install,” “link” and “connect” shall be comprehended in a broad sense, for example, it can be fixed connection, removable connection, or integral connection; and can also be directly connected, or indirectly connected through an intermediate medium, or connected inside two elements. For those of ordinary skill in the art, the specific implications of the above terms in the present application may be comprehended in accordance with specific conditions.

During tunnel construction, harmful gas often escapes or gushes out from the tunnel face or unsupported tunnel walls. When the concentration of harmful gas in the tunnel reaches a certain level, it can cause a series of disasters, such as explosions triggered by methane or hydrogen, poisoning caused by carbon monoxide, hydrogen sulfide, or sulfur dioxide, and suffocation due to carbon dioxide, threatening the safety of operators, delay in construction, and even major engineering accidents.

To mitigate the risk of harmful gases in tunnel construction, advance geological forecast method is typically employed before formal excavation of the tunnel face, to determine the hazards of harmful gases in the tunnel.

The existing methods for advanced detection of harmful gas in tunnel mainly include installing sensors at the drill hole opening, to detect the concentration of harmful gas. The sensors chiefly include two types: contact sensors and laser sensors based on laser absorption spectroscopy.

Due to the considerable depth of advance drilling hole, which can extend up to hundreds of meters, the concentration of harmful gas gushing from newly exposed strata at the opening may differ significantly from that at the bottom of the hole, which leads to substantial errors when using contact sensors to detect gas concentration at the opening.

Although laser sensors based on laser absorption spectroscopy can address the issue of long-distance detection of harmful gas, the concentration of harmful gas detected by such sensors represents an average concentration per meter (ppm·m), which means that even if the harmful gas concentration at the bottom of the hole is high, the detection results may not high, after averaging over hundreds of meters, resulting in lower accuracy.

Another existing method for advanced detection of harmful gas in tunnel involves sealing the drill hole for 24 hours after advance drilling and then measuring the harmful gas concentration in the hole; yet this method fails to detect the actual harmful gas concentration during excavation while causing low construction efficiency and hindering normal tunneling progress.

Based on the above, the embodiments of the present application provide a device and method for harmful gas detection in tunnel advance drilling, aiming to accurately detect the harmful gas concentration at the bottom of the hole, and provide reliable data for hazard assessment of harmful gas in tunnels. Harmful gases include, but not limited to, methane, hydrogen, carbon monoxide, hydrogen sulfide, sulfur dioxide, carbon dioxide, etc.

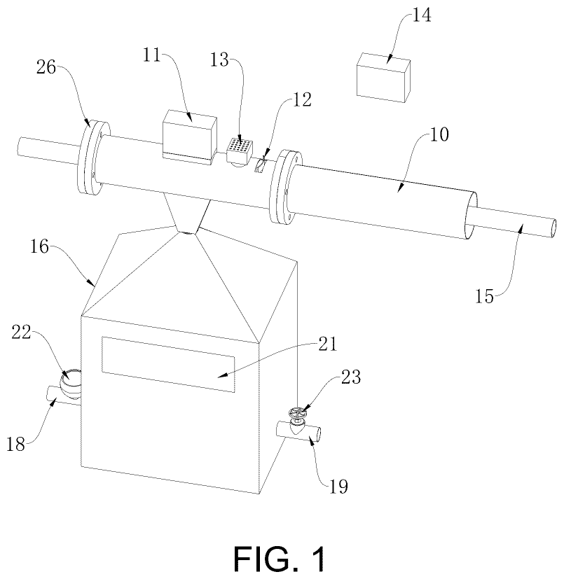

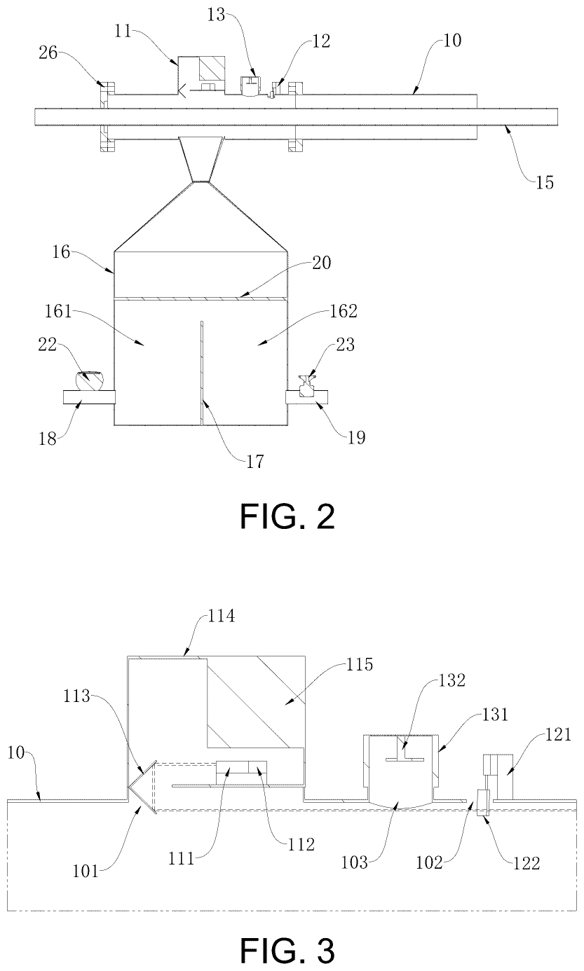

As shown in FIG. 1, FIG. 2 and FIG. 3, the embodiments of the present application provide a device for harmful gas detection in tunnel advance drilling, which comprises a casing pipe 10, and the casing pipe 10 is provided with a laser monitoring module 11, which is used for emitting laser axially in the casing pipe 10, ranging and monitoring harmful gas concentration; the casing pipe 10 is provided with an occlusion module 12 for periodically blocking laser emitted by the laser monitoring module 11; and the casing pipe 10 is provided with a gas flow rate monitoring module 13 for monitoring the velocity of harmful gas in the casing pipe 10.

As shown in FIG. 1 and FIG. 2, the casing pipe 10 is a structure with the left end closed and the right end open. During use, the open end of the casing pipe 10 is connected to the opening of the opening of the advance drilling hole 25 to guide the flow of harmful gas and groundwater from the advance drilling hole 25 into the casing pipe 10. Of course, the casing pipe 10 may also be a structure with openings at both ends, which is not limited herein.

For example, the casing pipe 10 is designed to fit over a drill rod 15 and can move axially along the drill rod 15. For example, the casing pipe 10 may be connected to the drill rod 15 via a flange assembly 26, for installation and support of the casing pipe 10 using the drill rod 15.

As shown in FIG. 1 and FIG. 2, the laser monitoring module 11 and the occlusion module 12 are arranged along the casing pipe 10 by axial spacing.

The laser emitted by the laser monitoring module 11 can propagate inside the casing pipe 10, with its direction parallel to the axial direction of the casing pipe 10, which can be used not only for ranging but also for monitoring harmful gas concentration based on the principle of laser absorption spectroscopy.

The occlusion module 12 can periodically block the laser along its propagation path inside the casing pipe 10. In other words, the occlusion module 12 can periodically and regularly block the laser, ensuring complete obstruction at specific time intervals.

The gas flow rate monitoring module 13 is installed on the casing pipe 10 to detect the flow rate of harmful gas entering the casing pipe 10. The gas flow rate monitoring module 13 may be positioned between the laser monitoring module 11 and the occlusion module 12.

The laser monitoring module 11 may include a laser gas sensor 111 and a laser ranging sensor 112 installed inside the casing pipe 10. The laser gas sensor 111 emits laser axially along the casing pipe 10 to monitor harmful gas concentration, while the laser ranging sensor 112 emits laser axially for ranging purposes.

As shown in FIG. 3, the laser monitoring module 11 includes a laser gas sensor 111, a laser ranging sensor 112, and a reflecting prism 113 installed outside the casing pipe 10. The wall of the casing pipe 10 is provided with a laser hole 101, and the reflecting prism 113 reflects the laser emitted by the laser gas sensor 111 and the laser ranging sensor 112 into the casing pipe 10 through the laser hole 101, propagating axially along the casing pipe 10. The laser gas sensor 111 is used to monitor harmful gas concentration, and the laser ranging sensor 112 is used for ranging, wherein the positions of the laser gas sensor 111 and the laser ranging sensor 112 in the axial direction of the casing pipe 10 are consistent. The dashed lines in FIG. 3 represent the propagation path of the laser.

Accordingly, by arranging the laser gas sensor 111, laser ranging sensor 112 and reflecting prism 113 outside the casing pipe 10 and providing a laser hole 101 in the pipe wall, it can make the entire laser monitoring module 11 more convenient to install and maintain, and also avoid occupying the installation space inside the casing pipe 10, to prevent groundwater and debris flowing to the casing pipe 10 from contacting and damaging the sensors during drilling, and extend the service life of the laser monitoring module 11.

As shown in FIG. 3, the laser monitoring module 11 further includes a monitoring box 114 connected to the casing pipe 10, with the laser gas sensor 111, laser ranging sensor 112, and reflecting prism 113 installed inside the monitoring box 114.

Accordingly, the monitoring box 114 provides physical protection for the laser gas sensor 111, laser ranging sensor 112, and reflecting prism 113, shielding them from dust, moisture, vibration, and other environmental factors that could cause damage, thereby extending their service life. The monitoring box 114 can also reduce external electromagnetic and optical interference, ensuring the accuracy and stability in measurement of the sensors. Integrating all components into a single monitoring box 114 makes the entire laser monitoring module 11 more compact, facilitating installation and maintenance while reducing the complexity and time required for on-site installation.

As shown in FIG. 3, the laser monitoring module 11 further includes a power supply 115 installed inside the monitoring box 114, connected to both the laser gas sensor 111 and the laser ranging sensor 112.

Accordingly, integrating the power supply 115 in the monitoring box 114 reduces the complexity of external power lines, simplifies wiring and installation, and avoids the issues associated with routing multiple power lines in external environments. This integrated power supply design also enhances the flexibility of the laser monitoring module 11, e.g. adding more sensors or other components only requires internal connections within the monitoring box 114, with no need for rewiring or installing external power supply.

The occlusion module 12 may be installed inside the casing pipe 10, to periodically block the laser by using flipping, moving, rotating and other functions of the occlusion module 12.

As shown in FIG. 3, the occlusion module 12 includes a driver 121 installed outside the casing pipe 10 and an occluder 122 connected to the driver 121. The wall of the casing pipe 10 is provided with an avoidance hole 102 to dodge the occluder 122. The driver 121 drives the occluder 122 to move and periodically block the laser inside the casing pipe 10. The movement of the occluder 122 may include moving, rotation, etc., without specific limitations.

Illustratively, the driver 121 may include a motor, and the occluder 122 may include a damper, with the output shaft of the motor connected to the damper. During operation, the output shaft of the motor rotates the damper, which periodically blocks the laser inside the casing pipe 10. The driver 121 may also include a linear actuator, and the occluder may include a damper, with the piston rod of the linear actuator connected to the damper. During operation, the extension and retraction of the piston rod drive the damper for reciprocating movement, thereby periodically blocking the laser inside the casing pipe 10.

Accordingly, integrating the occlusion module 12 outside the casing pipe 10 not only facilitates installation and maintenance, but also avoids occupying space inside the casing pipe 10.

The gas flow rate monitoring module 13 may include a gas flow rate sensor installed in the casing pipe 10. When harmful gas flows into the casing pipe 10, the gas flow rate sensor will measure its flow rate.

As shown in FIG. 3, the gas flow rate monitoring module 13 includes a flow rate monitoring box 131 installed outside the casing pipe 10 and a gas flow rate sensor 132 installed inside the monitoring box 131, and the monitoring box 131 is interconnected to the cavity of the casing pipe 10. For example, the top of the wall of the casing pipe 10 is designed with an interconnecting hole 103, and the flow rate monitoring box 131 is interconnected to the cavity of the casing pipe 10 through this hole. The top of the flow rate monitoring box 131 may be arranged with several overflow holes to allow harmful gas inside the casing pipe 10 to escape.

Accordingly, the gas flow rate sensor 132 can monitor the flow rate of harmful gas entering the casing pipe 10. Installing the sensor outside the casing pipe 10 can facilitate installation and maintenance, and avoid occupying space inside the casing pipe 10, to prevent groundwater and debris flowing to the casing pipe 10 from contacting and damaging the sensor during drilling, and extend the service life of the gas flow rate sensor 132. The flow rate monitoring box 131 also provides physical protection for the gas flow rate sensor 132, reducing external interference to the sensor and ensuring its stable and reliable measurements.

In some embodiments, as shown in FIG. 1, the detection device of the present application further includes a control module 14, connected to the laser monitoring module 11, occlusion module 12, and gas flow rate monitoring module 13. For example, the control module 14 may include PLC controller, computer, control center, etc.

Accordingly, the control module 14 can centrally control the laser monitoring module 11, the occlusion module 12, and the gas flow rate monitoring module 13, to achieve automated control, unified management, and coordinated operation of the entire detection device, thereby simplifying operations and reducing complexity and probability of errors. The control module 14 can also execute precise control algorithms based on sensor feedback data, to achieve high-precision control. The control module 14 can further integrate measurement data from various sensors for unified data processing and analysis, generating the comprehensive monitoring reports. For example, through control algorithms, it precisely calculates the concentration of harmful gas at the hole bottom and generates the harmful gas evaluation report.

In some embodiments, as shown in FIG. 1 and FIG. 2, a water tank 16 is arranged below the casing pipe 10 interconnected with its cavity; the bottom of the cavity of the water tank 16 is arranged with a water barrier 17 extending upward, forming a water outlet chamber 161 and a sampling chamber 162 on both sides of the water barrier 17. The lower end of the water tank 16 is provided with an outlet pipe 18 connected to the outlet chamber 161, and a sampling pipe 19 connected to the sampling chamber 162. Illustratively, the top of the water tank 16 is interconnected with the bottom of the casing pipe 10, the outlet pipe 18 is arranged with a flow meter 22, and the sampling pipe 19 is arranged with a sampling valve 23.

Correspondingly, the water tank 16 can collect groundwater gushing out from the advance drilling hole. The water barrier 17 divides the water tank 16 into the water outlet chamber 161 and the sampling chamber 162, preventing interference between the water outlet and sampling processes. The water outlet chamber 161 directly discharges water through the outlet pipe 18, ensuring smooth and efficient water discharge. The flow meter 22 measures the flow rate of the groundwater. The sampling chamber 162 is dedicated to sampling. By opening the sampling valve 23, samples can be taken through the sampling pipe 19 for analyzing the content of soluble harmful gases in the groundwater.

As shown in FIG. 1 and FIG. 2, a horizontally arranged filter 20 is located above the water barrier 17 in the water tank 16, and the water tank 16 is provided with a slag discharge door 21 for opening and closing the cavity above the filter 20. Illustratively, the filter 20 can be a grid, screen, etc.

Correspondingly, the filter 20 can remove drill slags entering the water tank 16 with the groundwater, preventing them from entering the water outlet chamber 161 and the sampling chamber 162, and clogging the outlet pipe 18 and sampling pipe 19. The slag discharge door 21 is so designed as to easily open the cavity above the filter 20, facilitating regular cleaning of impurities and deposits on the filter 20, thereby reducing maintenance time and costs.

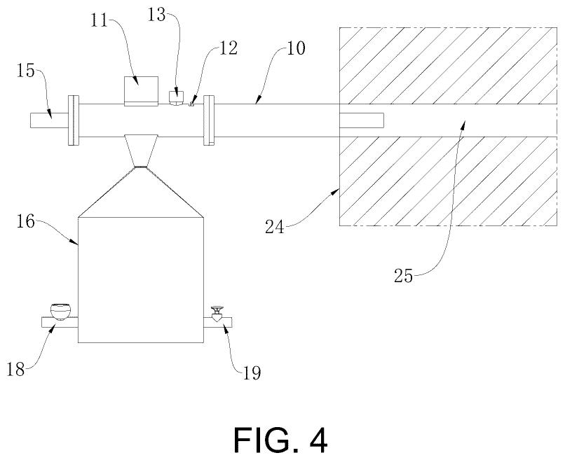

As shown in FIG. 4, the embodiment of the present application also provides a method for harmful gas detection in tunnel advance drilling, using the device for harmful gas detection in tunnel advance drilling, which comprises:
S1: Connect one end of the casing pipe 10 to the opening of the advance drilling hole opening 25.

Illustratively, after drilling according to the advance geological drilling design requirements, the casing pipe 10 is put on the drill rod 15 and placed between the tunnel face 24 and the drilling machine, with the right end of the casing pipe 10 connected to the opening of the advance drilling hole 25, and the left end connected to the drill rod 15 via a flange assembly 26. This can direct groundwater and harmful gas from the advance drilling hole 25 into the casing pipe 10. For simplicity, the structure of the drilling machine is not shown in FIG. 4.
S2: The laser monitoring module 11 emits laser beam to the bottom of the advance drilling hole 25 and obtains the harmful gas concentration cl from the laser monitoring module 11 to the bottom of the advance drilling hole 25.

Illustratively, the laser gas sensor 111 emits laser to the bottom of the advance drilling hole 25 and obtains the harmful gas concentration cl (ppm·m) from the laser gas sensor 111 to the bottom of the advance drilling hole 25. Additionally, the laser ranging sensor 112 emits laser beam to the bottom of the advance drilling hole 25 and obtains the distance dl (m) from the laser ranging sensor 112 to the bottom of the advance drilling hole 25.
S3: After a time interval tl, the occlusion module 12 blocks laser beam inside the casing pipe 10. The laser monitoring module 11 emits laser beam to the occlusion module 12 and obtains the harmful gas concentration c2 and the distance d2 from the laser monitoring module 11 to the occlusion module 12. The gas flow rate monitoring module 13 measures the flow rate v of harmful gas inside the casing pipe 10.

Illustratively, after a time interval tl, the occlusion module 12 blocks the laser beam, preventing it from propagating to the bottom of the advance drilling hole 25. The laser gas sensor 111 emits laser to the occlusion module 12 and obtains the harmful gas concentration c2 (ppm·m) from the laser gas sensor 111 to the occlusion module 12. The laser ranging sensor 112 emits laser beam to the occlusion module 12 and obtains the distance d2 (m) from the laser ranging sensor 112 to the occlusion module 12. The gas flow rate sensor 132 measures the flow rate v (m/s) of harmful gas in the casing pipe 10.
S4: After a time interval t2, the occlusion module 12 releases laser beam inside the casing pipe 10. The laser monitoring module 11 emits laser beam to the bottom of the advance drilling hole 25 and obtains the harmful gas concentration c3 from the laser monitoring module 11 to the bottom of the advance drilling hole 25.

Illustratively, after a time interval t2, the occlusion module 12 moves away, allowing the laser beam to propagate to the bottom of the advance drilling hole 25. The laser gas sensor 111 emits laser to the bottom of the advance drilling hole 25 and obtains the harmful gas concentration c3 (ppm·m) from the laser gas sensor 111 to the bottom of the advance drilling hole 25.

Since the occlusion module 12 periodically blocks laser beam, the occlusion cycle is set to T(s), and T=tl+t2.

As harmful gas at the bottom of the advance drilling hole 25 flows toward the opening along the drilling hole, the distance d3 (m) traveled by harmful gas inside the hole during the cycle T can be calculated as follows: d3=v×T.

Due to the continuous flow of harmful gas inside the advance drilling hole 25, some harmful gas flows outside the casing pipe 10. The harmful gas concentration c2 obtained by the laser gas sensor 111 from the laser monitoring module 11 to the occlusion module 12 can be regarded as the average concentration of harmful gas flowing toward the outside of the casing pipe 10. The concentration c4 (ppm·m) of harmful gas flowing out of the casing pipe 10 can be calculated with the following formula: c4=(c2/d2)·d3.
S5: Based on the data obtained in Steps S2, S3, and S4, the concentration of harmful gas at the bottom of the advance borehole 25 is calculated.

During the cycle T, harmful gas inside the advance drilling hole 25 flows out and is also replenished. By subtracting the harmful gas concentration obtained in Step S1 from that obtained in Step S3 and adding the concentration of harmful gas flowing out of the casing pipe 10, the average concentration of harmful gas gushed from the newly exposed stratum at the bottom of the advance drilling hole 25 over the distance d3 can be obtained.

The concentration C (ppm) of harmful gas gushing from the newly exposed stratum at the bottom of the advance drilling hole 25 can be calculated with the following formula: C=(c3−c1+c4)/d3=[c3−c1+(vTc2/d2)]/(vT).

The device and method for harmful gas detection in tunnel advance drilling provided by the embodiments of the present application enable real-time and accurate detection of harmful gas concentration at the bottom of advance drilling hole while going with the drill rod 15 during drilling operations, to provide precise data for hazard assessment of harmful gases in tunnel.

### Embodiment

During the construction of a tunnel, methane was detected, necessitating measurement of methane concentration in unexcavated sections for gas classification and harmful gas evaluation.

When using the device for harmful gas detection in tunnel advance drilling provided in the embodiments of the present application for detection, with the occlusion cycle of the occlusion module (12 of T=2 s, the detected data are: cl=200 ppm·m, c2=100 ppm·m, c3=220 ppm·m, v=0.1 m/s, d2=0.2 m.

The methane gas concentration from newly exposed strata at the bottom of the advance drilling hole 25 is calculated as:

C=[220−200+(0.1×2×100)/(0.1×2)]/(0.1×2)=600 ppm

The conventional detection methods can only detect an average methane concentration of 200 ppm·m to 220 ppm·m in the hole. This demonstrates that the conventional methods are inadequate for accurately detecting harmful gas concentration at the drilling hole bottom.

During drilling, groundwater and cooling water of the drilling machine enter the water tank 16 through the casing pipe 10. Inspectors collect water samples via the sampling pipe 19 for testing soluble harmful gas content in the groundwater.

The above is only a specific implementation of the present application, but the protection scope of the present application is not limited hereto. Any technician familiar with the technical field can easily think of various changes or replacements within the technical scope disclosed by the present application, which should be covered by the protection scope of the present application.

What is claimed is:

1. A device for harmful gas detection in tunnel advance drilling, wherein the device comprises a casing pipe, the casing pipe is arranged with a laser monitoring module for emitting laser axially in the casing pipe, ranging and monitoring a harmful gas concentration;
the casing pipe is designed with an occlusion module for periodically blocking laser emitted by the laser monitoring module; the casing pipe is arranged with a gas flow rate monitoring module for monitoring a flow rate of a harmful gas within the casing pipe.

2. The device for harmful gas detection in tunnel advance drilling according to claim 1, wherein the laser monitoring module comprises a laser gas sensor, a laser ranging sensor, and a reflecting prism arranged outside the casing pipe, a wall of the casing pipe is provided with a laser hole, and the reflecting prism reflects laser emitted by the laser gas sensor and the laser ranging sensor into the casing pipe for axial propagation along the casing pipe via the laser hole.

3. The device for harmful gas detection in tunnel advance drilling according to claim 2, wherein the laser monitoring module further comprises a monitoring box connected to the casing pipe, and the laser gas sensor, the laser ranging sensor, and the reflecting prism are arranged inside the monitoring box.

4. The device for harmful gas detection in tunnel advance drilling according to claim 1, wherein the occlusion module comprises a driver arranged outside the casing pipe and an occluder connected to the driver, a wall of the casing pipe is provided with an avoidance hole for dodging the occluder, and the driver drives the occluder to move and periodically block laser in the casing pipe.

5. The device for harmful gas detection in tunnel advance drilling according to claim 1, wherein the gas flow rate monitoring module comprises a flow rate monitoring box arranged outside the casing pipe and a gas flow rate sensor inside the flow rate monitoring box, and the flow rate monitoring box is interconnected with a cavity of the casing pipe.

6. The device for harmful gas detection in tunnel advance drilling according to claim 1, wherein the device further comprises a control module connected to the laser monitoring module, the occlusion module and the gas flow rate monitoring module.

7. The device for harmful gas detection in tunnel advance drilling according to claim 1, wherein the casing pipe is put on a drill rod and can move axially along the drill rod.

8. The device for harmful gas detection in tunnel advance drilling according to claim 1, wherein a water tank interconnecting with its cavity is arranged below the casing pipe; a bottom of the cavity of the water tank is provided with a water barrier extending upward, forming a water outlet chamber and a sampling chamber on both sides of the water barrier, a lower end of the water tank is provided with an outlet pipe connected to the outlet chamber, and a sampling pipe connected to the sampling chamber.

9. The device for harmful gas detection in tunnel advance drilling according to claim 8, wherein a horizontally arranged filter is located above the water barrier in the water tank, and the water tank is provided with a slag discharge door for opening and closing the cavity above the filter.

10. A method for harmful gas detection in tunnel advance drilling, wherein a device for harmful gas detection in tunnel advance drilling according to claim 1 is adopted, the method comprises:
S1: one end of the casing pipe is connected to an opening of an advance drilling hole;
S2: the laser monitoring module emits laser to a bottom of the advance drilling hole and obtains a harmful gas concentration cl from the laser monitoring module to the bottom of the advance drilling hole,
S3: after a time interval tl, the occlusion module blocks a laser beam inside the casing pipe, The laser monitoring module emits laser to the occlusion module and obtains a harmful gas concentration c2 and a distance d2 from the laser monitoring module to the occlusion module, the gas flow rate monitoring module measures the flow rate v of harmful gas inside the casing pipe,
S4: after a time interval t2, the occlusion module releases laser inside the casing pipe, the laser monitoring module emits laser to the bottom of the advance drilling hole and obtains a harmful gas concentration c3 from the laser monitoring module to the bottom of the advance drilling hole,
S5: based on a data obtained in Steps S2, S3, and S4, the concentration of the harmful gas at the bottom of the advance drilling hole is calculated.
